(19) United States
(12) Patent Application Publication
(10) Pub. No.: US 2011/0177018 A1
(43) Pub. Date: Jul. 21, 2011

(54) Personal Care Compositions Comprising A Hydrophobically Modified Cationic Polysaccharide

(76) Inventors: Paul Martin Lipic, West Chester, OH (US); Timothy Woodrow Coffindaffer, Maineville, OH (US); Nicole Lynette Westall, Mason, OH (US); Kenneth Eugene Kyte, III, Oregonia, OH (US); Benjamin Parker Heath, Cincinnati, OH (US)

(21) Appl. No.: 13/005,012

(22) Filed: Jan. 12, 2011

Related U.S. Application Data
(60) Provisional application No. 61/295,455, filed on Jan. 15, 2010.

(51) Int. Cl. A61K 8/73; A61Q 9/02; A61K 8/87; A61K 8/88; A61K 8/81

(52) U.S. Cl. 424/70.13

(57) ABSTRACT

A personal care composition which is preferably a post-foaming gel, said composition comprising a cationic polysaccharide which is hydrophobically modified with a hydrophobic substituent and a cationic substituent; a water dispersible surface active agent, of a carrier comprising water; and optionally a lubricant.

### CROSS REFERENCE TO RELATED APPLICATIONS

This application claims the benefit of U.S. Provisional Application No. 61/295,455 filed on Jan. 15, 2010.

### FIELD OF THE INVENTION

The present invention relates to a personal care composition, preferably one in the form of an aerosol shaving gel or foam, or a non-aerosol composition, which contains a cationic polysaccharide and multiple types of lubricants. The personal care composition is preferably a shave or hair removal preparation.

### BACKGROUND OF THE INVENTION

One of the more popular forms used today is the post foaming shave gel, developed in the late 70's. Amine-based soaps are combined with volatile hydrocarbons to form a clear, stable emulsion when kept under pressure. Once dispensed and mechanically agitated, these gels transform into thick foams.

Currently, a widely used form of shaving preparation is the type referred to as a post-foaming shave gel. These post-foaming shave gels are now well-known. See, e.g., U.S. Pat. Nos. 5,326,556 and 5,500,211. Various attempts have been made to increase the lubricity of shaving compositions. The addition of various polymers into personal care compositions is known. See e.g. U.S. Patent Publ. No. 2007/0207106; U.S. Pat. Nos. 5,902,574 and 5,262,154. Further, in some cases a lubricious water soluble polymer such as polyethylene oxide or polyvinylpyrrolidone has been added. See, e.g., U.S. Pat. Nos. 5,560,859 and 5,858,343. In other cases, water insoluble particles have been added, including water insoluble polymer particles, such as polytetrafluoroethylene, polyethylene, or polyamide (nylon) particles, and water insoluble inorganic particles such as titanium dioxide or glass beads. See, e.g., U.S. Pat. Nos. 5,587,156 and 4,155,870. Various other shave gels have been disclosed. See, e.g., U.S. Patent Publ. No. 2006/0257349, 2006/0257350 and 2005/0175575 and U.S. Pat. Nos. 5,500,211 and 6,352,689.

Despite the addition of various cationic polymers into various shave preparations, there is an ongoing need for new polymers which can provide lubrication for a personal care composition, such as a post-foaming gel composition.

### SUMMARY OF THE INVENTION

One aspect of this invention relates to a personal care composition which is in the form of an aerosol product, preferably a post foaming gel or a shaving foam, said composition comprising: from about 0.005% to about 3% of a cationic polysaccharide, wherein said cationic polysaccharide is hydrophobically modified with a hydrophobic substituent and a cationic substituent; from about 2% to about 25% of a water dispersible surface active agent; from about 60% to about 93% of a carrier comprising water; and a lubricant.

### DETAILED DESCRIPTION OF THE INVENTION

The term “fatty”, as used herein, means a hydrocarbon chain having 12-22 carbon atoms (C12-22), preferably 14-18 carbon atoms (C14-18). The chain may be straight or branched and may be saturated or unsaturated (typically one or two double bonds in the chain). The term “water dispersible”, as used herein, means that a substance is either substantially dispersible or soluble in water.

The personal care composition of the present invention is suitable for use as a hair removal preparation, such as a post-foaming shave gel composition. In one embodiment the composition comprises from about 0.005% to about 3% of a cationic polysaccharide, wherein said cationic polysaccharide is hydrophobically modified; about 2% to about 25%, preferably about 5% to about 20%, of a water dispersible surface active agent, from about 60% to about 93%, or from about 70% to about 85% of a carrier, such as water; and a lubricant. The lubricant can comprise preferably about 0.01% to about 1%, lubricious water soluble polymer, about 0.01% to about 5%, preferably about 0.1% to about 2%, water insoluble particles, and about 0.0005% to about 3%, preferably about 0.001% to about 0.5%, hydrogel-forming polymer, by weight of the composition. Preferably, the composition is in the form of a post-foaming shave gel and will additionally include about 1% to about 6%, preferably about 2% to about 5%, volatile post-foaming agent.

### 1. Hydrophobically Modified Cationic Polysaccharide

The personal care composition of the present invention comprises a hydrophobically modified cationic polysaccharide, modified with a hydrophobic substituent and a cationic substituent. The hydrophobically modified cationic polysaccharide is used at a level of from about 0.005% to about 3%, or from about 0.01% to about 2.0%, or from about 0.02 to about 1%, or from about 0.025% to about 0.5%, by weight. Non-limiting examples of suitable hydrophobically modified cationic polysaccharides comprise cellulose, starch and guar derivatives, particularly a derivatized hydroxyethyl cellulose ether (such as those sold under the Trade Name of SoftCAT™).

Nonlimiting examples of hydrophobically modified quaternized hydroxyethyl cellulose ethers include: those referred to in US 2007 0031362 A1 from Union Carbide, and can be referred to by those skilled in the art as SoftCAT.

“Hydrophobically modified quaternized cellulose ethers” refers to cellulose ether derivatives containing quaternary ammonium groups. In one embodiment, the quaternized cellulose ether comprises a cellulose ether which has from 4,000 to 10,000 anhydroglucose repeat units and which is substituted with

(a) on the average from 0.0003 to 0.08 moles, per mole of anhydroglucose unit, of a substituent comprising an alkyl or arylalkyl group having from 8 to 24 carbon atoms and

(b) a substituent having the formula II

[R⁵R⁶R⁷R⁸N⁺](A^{z-})_{1z} (II)

wherein
R⁵, R⁶ and R⁷ each independently are —CH₃ or —C₂H₅,
R⁸ is —CH₂—CHOH—CH₂— or —CH₂CH₂—
A^{z-} is an anion, and
z is 1, 2 or 3.

Preferably, these cellulose ethers have an M.S. (hydroxyethyl) of from 1.0 to 3.0, more preferably from 1.5 to 2.5. The M.S. (hydroxyethyl) designates the average number of moles of hydroxyethyl groups which have been attached by an ether linkage per mole of anhydroglucose unit. The cellulose ethers have at least 4,000 anhydroglucose repeat units, preferably at least 4,500 anhydroglucose repeat units, more preferably at least 5,000 anhydroglucose repeat units, and most preferably at least 6,000 anhydroglucose repeat units. The cellulose ethers have up to 10,000 anhydroglucose repeat units, preferably up to 9,000 anhydroglucose repeat units and most preferably up to 8,000 anhydroglucose repeat units. Such cellulose ethers are readily commercially available. Alternatively, such cellulose ethers can be prepared from cellulose by methods known in the art.

Typical cellulose ethers include for example, hydroxyethyl cellulose, hydroxypropyl cellulose, methyl cellulose, hydroxypropyl methyl cellulose, hydroxyethyl methyl cellulose, hydroxyethyl carboxylmethyl cellulose, or mixtures thereof. Preferred cellulose ethers include hydroxyethyl cellulose and hydroxypropyl cellulose. Other suitable cellulose ethers comprise hydroxyethyl groups. The above cellulose ethers can be derivatized with a hydrophobic substituent and a cationic nitrogen-containing substituent to form quaternized cellulose ethers of the present invention.

The cellulose ether derivatives of the present invention are cellulose ethers which are substituted with a hydrophobic substituent (a) and a cationic substituent (b) as described below.

Hydrophobic substituents (a) suitable for use in accordance with the present invention comprise an alkyl or arylalkyl group having from 8 to 24 carbon atoms, preferably from 10 to 24 carbon atoms, more preferably from 12 to 18 carbon atoms, and most preferably 12 to 15 carbon atoms. As used herein the term “arylalkyl group” means a group containing both aromatic and aliphatic structures. The most preferred aliphatic hydrophobic substituent is the dodecyl group, which is most preferably straight-chained. The hydrophobic substituent is typically cationic or non-ionic. Many hydrophobe-containing reagents suitable for use as hydrophobic substituents are commercially available. In addition, methods for preparing such hydrophobe-containing reagents, as well as methods for derivatizing cellulose ethers to comprise such hydrophobic substituents, are known to those skilled in the art. Note, for example, U.S. Pat. Nos. 4,228,277, 4,663,159 and 4,845,175.

A preferred hydrophobic substituent (a) suitable for use in accordance with the present invention has the formula (I)

R¹R²R³R⁴N⁺(A^{z-})_{1/z} (I)

wherein
R¹ and R² each independently are —CH₃ or —C₂H₅,
R³ is —CH₂—CHOH—CH₂— or —CH₂CH₂—
R⁴ is an alkyl or arylalkyl group having from 8 to 24 carbon atoms, and
A^{z-} is an anion and
z is 1, 2 or 3.

Preferably, R¹ and more preferably, both R¹ and R² are —CH₃. Preferably, R³ is —CH₂—CHOH—CH₂—. Preferably, R⁴ is —CₙH₍₂ₙ₊₁₎, where n is from 8 to 24, more preferably from 10 to 18, most preferably 12. A^{z-} is an anion with the valency of z, such as phosphate, nitrate, sulfate or halide. Chloride is the most preferred ion. Z is preferably 1 or 2, more preferably 1. The most preferred hydrophobic substituents (a) are those wherein two or more, preferably each of R¹, R², R³, R⁴, A^{z-} and z have the mentioned preferred meanings.

Other preferred hydrophobic substituents include those derived from hydrophobe-containing reagents comprising alkyl or arylalkyl groups having from 8 to 24 carbon atoms, preferably from 10 to 24 carbon atoms, more preferably from 12 to 18 carbon atoms, and most preferably 12 to 15 carbon atoms. Preferred are glycidyl ethers, such, as nonylphenyl glycidyl ether or dodecylphenyl glycidyl ether; or alpha-olefin epoxides, such as 1,2-epoxy hexadecane and their respective chlorohydrins, or alkyl halides, e.g., dodecyl bromide, and mixtures thereof.

The average substitution level of the substituent (a) is at least 0.0003, preferably at least 0.0005 moles per mole of anhydroglucose unit and up to 0.08, preferably up to 0.07, and most preferably up to 0.05 moles per mole of anhydroglucose unit. More than one particular hydrophobic substituent can be substituted onto the cellulose ether provided that the total substitution level is within the ranges set forth above.

The cationic substituent (b) suitable for use in accordance with the present invention has the formula II (above). Preferably, R⁵ is CH₃. More preferably, R⁵, R⁶ and R⁷ are —CH₃. Preferably, R⁸ is —CH₂—CHOH—CH₂—. A^{z-} is an anion with the valency of z, such as phosphate, nitrate, sulfate or halide. Chloride is the most preferred ion. Z is preferably 1 or 2, more preferably 1. The most preferred cationic substituents (b) are those wherein two or more, preferably each of R⁵, R⁶, R⁷, R⁸, A^{z-} and z have the mentioned preferred meanings

Preferred quaternized cellulose polymers with hydrophobic substitution are referred to in the industry Personal Care Products Council (formerly the Cosmetic, Toiletry, and Fragrance Association) as Polyquaternium-67 (PQ67) and are available from Dow Chemical (Amerchol Corp.) under the tradename SoftCAT™ which includes their SL, SX, and SK series polymers.

### 2. Water Dispersible Surface Active Agent

The water dispersible surface active agent is preferably one that is capable of forming a lather and may comprise a soap, an interrupted soap, a detergent, an anionic surfactant, a non-ionic surfactant or a mixture of one or more of these. One group of suitable water dispersible surface active agent are lathering surfactants, such as those selected from the group consisting of anionic surfactants, nonionic surfactants, amphoteric surfactants, zwiterrionic surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants

Concentrations of these surfactants are from about 1% to about 20%, alternatively from about 5% to about 25%, and alternatively from about 2% to about 30% by weight of the composition.

Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth-20, Steareth-21, Ceteth-20, Laureth-4 and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Suitable amphoteric surfactants include, for example, the betaines and sultaines such as cocoamidopropyl betaine, coco dimethyl carboxymethyl betaine, coco sultaine and the like. In one embodiment, the amphoteric surfactant is a betaine selected from consisting of coco betaine, lauryl amido betaine, or a mixture thereof, and reduced salt versions thereof. Non-limiting examples of suitable zwitterionic or amphoteric surfactants are described in U.S. Pat. Nos. 5,104,646 and 5,106,609.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. Suitable anionic lathering surfactants include, for example, the sodium, potassium, ammonium and substituted ammonium salts (such as the mono-, di- and triethanolamine salts) of C₈-C₂₂, preferably C12-C18, alkyl sulfates (e.g., sodium lauryl sulfate, ammonium lauryl sulfate), alkyl sulfonates (e.g., ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g., ammonium laureth sulfate).

The water dispersible surface active agent can also include soaps, such as the sodium, potassium and lower alkanolamine (preferably triethanolamine) salts of C12-22, preferably C14-18, fatty acids. Typical fatty acids include lauric, myristic, palmitic and stearic acid and mixtures thereof. The preferred fatty acids are palmitic and stearic. The interrupted soaps include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition must contain sufficient base to neutralize or partially neutralize the soap component and adjust the pH to the desired level (typically between 5 and 10, more typically between 6 and 9). It is most preferred that the composition of the present invention includes a soap (e.g., triethanolamine palmitate/stearate) or an interrupted soap (e.g., triethanolamine stearoyl/myristoyl sarcosinate), or a mixture thereof.

In one embodiment, the composition is free or essentially free of soap. As used herein, “essentially free” of a component means that no amount of that component is deliberately incorporated into the composition. In one embodiment the composition is a self-foaming soap free shave gel as described in U.S. Pat. No. 5,500,211.

### 3. Lubricant

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Particularly suitable polyethylene oxides include, for example, PEG-14M (MW≅600,000) PEG-23M (MW=1 million), PEG-45M (MW=2 million) and PEG-90M (MW=4 million). The lubricious water soluble polymer will generally be included in the post foaming gel composition in an amount of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles. Any of the forgoing particles may also include a surface treatment to make the particles more readily dispersible or improve their cosmetic aesthetics. Preferred are polytetrafluoroethylene particles (e.g., PTFE particles available from MicroPowders, Inc. under the tradename Microslip). Preferably the water insoluble particles will have an average particle size of about 1 μm to about 100 μm, more preferably about 2 μm to about 50 μm, and most preferably about 5 μm to about 15 μm. The particles may be of any desired shape including spherical bead, elongated fiber or irregular shape, with spherical bead being the preferred shape. Generally the water insoluble particles will be included in the post foaming gel composition in an amount of about 0.01% to about 5%, preferably about 0.1% to about 2%, by weight.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. Generally, the hydrogel-forming polymer will be included in the post foaming gel composition in an amount of about 0.0005% to about 3%, preferably about 0.001% to about 0.5%, more preferably about 0.002% to about 0.1%, by weight.

A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin A preferred source of glyceryl acrylate/acrylic acid copolymer is available from ISP Technologies, Inc. (United Guardian Inc.) under the tradename Lubrajel®, particular the form known as Lubrajel® oil which contains about 1.0%-1.3% glyceryl acrylate/acrylic acid copolymer in aqueous glycerin (^{˜}40% glycerin). Lubrajel® oil also includes about 0.6% PVM/MA copolymer (also known as methoxyethylene/maleic anhydride copolymer), which may further contribute to the lubricity of this source. Most preferably, the post foaming gel composition will include about 0.25% to about 4% Lubrajel® oil in order to provide a preferred level of about 0.002% to about 0.05% of the glyceryl acrylate/acrylic acid copolymer. This amount of Lubrajel® oil will also provide about 0.001% to about 0.03% of PVM/MA copolymer.

### 4. Post Foaming Agent

The post-foaming agent, when included in the post foaming gel composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig. The post-foaming agent will be present in an amount to provide the post foaming gel composition with a sufficiently rapid turnover—that is, transition from gel to foam when contacted with the skin—typically, in about 2 to about 30 seconds, preferably in about 5 to about 15 seconds.

### 5. Carrier

The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the post foaming gel composition comprises from about 50% to about 99.99%, preferably from about 60% to about 93%, more preferably from about 70% to about 90%, and even more preferably from about 80% to about 85% of the carrier by weight of the composition. In one embodiment, the carrier comprises water.

### 6. Other Adjunct Ingredients

Although not necessary to forming a useful shave gel composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, etc. It is particularly preferred to include glycerin in the shave gel composition of the present invention, preferably in an amount of about 0.1% to about 3%, more preferably about 0.3% to about 1%, by weight. Glycerin improves the emolliency of the composition.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); Crosslinked Polyacrylate Polymers (including both cationic and nonionic polymers, such as described in U.S. Pat. Nos. 5,100,660; 4,849,484; 4,835,206; 4,628,078; 4,599,379, and EP 228,868); Polymeric sulfonic acid (such as copolymers of acryloyldimethyltaurate and vinylpyrrolidone) and hydrophobic ally modified polymeric sulfonic acid (such as crosspolymers of acryloyldimethyltaurate and beheneth-25 methacrylate); Polyacrylamide Polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); Polysaccharides (nonlimiting examples of polysaccharide gelling agents include those selected from the group consisting of cellulose, carboxymethyl hydroxyethylcellulose (sold under the trademarks “Natrosol”), cellulose acetate propionate carboxylate, hydroxyethylcellulose, hydroxyethyl ethylcellulose, hydroxypropylcellulose (sold under the trademarks “Klucel”), hydroxypropyl methylcellulose, methyl hydroxyethylcellulose, microcrystalline cellulose, sodium cellulose sulfate, and mixtures thereof); Gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. Nos. 6,967,027 to Heux et al.; 5,207,826 to Westland et al.; 4,487,634 to Turbak et al.; 4,373,702 to Turbak et al. and 4,863,565 to Johnson et al., U.S. Patent Publ. No. 2007/0027108 to Yang et al.)

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof. Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and US 2005/0019356 (i.e. desquamation actives, anti-acne actives, chelators, flavonoids, and antimicrobial and antifungal actives). Examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed. Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The composition can also include other commonly included ingredients which are used in commercially available post foaming shave gels such as those described in US Patent Publ. Nos. 2006/0257349, 2006/0257350 and 2005/0175575.

### 7. Product Forms and Uses

The personal care compositions of the present invention can be used for as a hair removal preparation such as a post foaming shave gel. The present composition may be formulated as an aerosol foam, a post-foaming gel (which is the preferred form) or a non-aerosol gel or lather. It may be packaged in any suitable dispenser normally used for dispensing personal care compositions (such as shaving compositions). These include collapsible tubes, pump or squeeze containers, and aerosol-type dispensers, particularly those with a barrier to separate the post foaming gel composition from the propellant required for expulsion.

The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) manually activated air pump spray devices in which a pump system is integrated into the container to allow the user to pressurize the container with air in order to expel the product (e.g., the “AIRSPRAY” system available from Airspray International); (3) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (4) bag-in-can (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

### 8. In Shave Lubrication Test

It has been found that the personal care composition of the present invention provides for an in shave lubrication benefit as shown by reduced friction as measured by the In Shave Lubrication “ISL” Test defined herein. Reducing friction is important during the shave because a high friction skin surface results in bulging of the skin When the skin bulges, the blade is more likely to engage the skin, increasing the chance for skin irritation. Therefore, by reducing friction the product helps protect the skin In addition, lower friction results in less drag on the skin, which can also be a potential source of irritation. This method enables measurement of the coefficient of friction (CoF) of a shave preparation.

In Shave Lubrication Test Method: An apparatus designed to simulate lubrication during the shaving process is connected to an instrument capable of measuring frictional forces (for example, an Instron-type instrument) and containing a load cell of about 1 kg to about 100 kg. The rinsing apparatus comprises: 1) an air-activated clamping device capable of opening and closing to deliver pressures of about 10 psi to about 70 psi to simulate the pressure exerted by hands on hair during rinsing 2) keratinous tissue models as described herein affixed to two opposing sides of the clamping device and 3) one or more spray nozzles capable of delivering water flow rates of from about 50 ml/min. to about 1000 mL/min., for simulating shower conditions.

Procedure: Attach the rinsing apparatus to the base of a Stable Micro Systems TA XT Plus™ Texture Analyzer (TA) equipped with a 30 kg load cell, centering or aligning the clamps perpendicular to the load cell. Adjust water flow rate to approximately 200 ml/min and the water temperature to 103° F.+/−2° F. Set the air pressure for the TA clamps to approximately 30 psi. Set the instrument measurement settings as follows: TA settings, tension compression, test speed—10.0 mm/sec for 130 mm long pull. Set the macro for a total of 10 strokes. Run the first five strokes without the water on, then manually turn on the water for 2 min 15 sec for the second five strokes. During the test, data (g of force) will only be collected during the upward pull of the treated KTM, not on the return. Cover the pads on both the front and back side of the piston with a polyurethane skin pad (see JP2006233367 for details).

Wet a 2 inch by 9 inch piece of nonwoven KTM under hot (˜103° F.+/−2° F.) tap water for 30 seconds. Place 2 g+/−0.1 g of aerosol shave gel or 1 g+/−0.1 g of aerosol shave foam onto the nonwoven KTM and gently lather and spread by hand for 30 seconds. Rub excess foam on the back of the nonwoven KTM. Load the nonwoven KTM into the TA and start the test macro. At the end of the fifth stroke, turn on the rinse water. Initiate a test sequence which 1) instructs the instrument to raise the load cell to which the KTM is attached, at a rate of about 10 mm/sec 2) opens the clamps, and 3) instructs the instrument to lower the load cell. Repeat this sequence until a predetermined number of sequences may be executed, for example, 10. Between each sample, wipe the polyurethane skin pads with a piece of nonwoven KTM and an alcohol wipe to remove any possible build-up from the previous test. By calculating the total friction in grams of force (or other suitable unit of force) for dry friction and rinse friction, products may be ranked relative to each other to assess which products would be expected to have the most pleasant feel.

“KTM” as defined herein means a “Keratinous tissue mimic” which refers to one or more artificial substrates which may have one or more physical properties representative of keratinous tissue. The KTM used for the purposes of this application is TENCEL from Lenzing, Inc Additional details on other KTMs is disclosed in Section I of U.S. Ser. No. 61/239,908 to Battaglia et al, filed Sep. 4, 2009.

### 9. Methods of Making

Those of ordinary skill in the art will understand that the personal care composition can be made in a variety of known ways for making similar compositions. In one embodiment, where the personal care composition is a aerosol, such as a post foaming shave gel, the method of making is as described in U.S. Patent Publ. no. 2006/0257349, paragraph 21.

The water soluble polymers (such as the hydrophobically modified cationic polysaccharide of the present invention, polyethylene oxide, hydroxyethylcellulose) are added to water and mixed until the polymers are completely dissolved (about 30 min) The aqueous mixture is then heated and the glyceryl oleate, sorbitol and fatty acids are added at about 60° C. and well mixed while the heating continues. At 80-85° C. the triethanolamine is added and mixed for about 20 minutes to form the aqueous soap phase. After cooling the aqueous soap phase to room temperature, the remaining components (i.e., Lubrajel, glycerin, fragrance, colorant, botanicals) are added to the aqueous soap phase and mixed well to form the gel concentrate. (Water may be added if required to bring the batch weight to 100%, thereby compensating for any water loss due to evaporation.) The concentrate is then combined with the volatile post-foaming agent under pressure within the filling line and filled into bottom-gassed aerosol cans with shearing through the valve under nitrogen pressure.

In one embodiment, the present invention comprises a method of making the personal care composition comprising the steps of: forming a first premix comprising water and a water soluble polymers, said water soluble polymer comprising a hydrophobically modified cationic polysaccharide, and optionally a polyethylene oxide, hydroxyethylcellulose, or a mixture thereof. The first premix is preferably formed at an elevated temperature such as from about 60 C to about 85 C, preferably at about 75 C. The method further comprises a step of forming at least a second premix comprising a hydroxypropylmethylcellulose, a base suitable for use in a personal care composition (including but not limited to TEA, NaOH, KOH, mixtures thereof) and water. The step of forming the second premix can be done at an elevated temperature or at room temp. method can further comprise yet another step of combining said first premix and said second premix, preferably while maintaining, a temperature of about 60 C to about 85 C, preferably at about 75 C. During this mixing step any solid or waxy ingredients (such as fatty acids) can be added. The combined mixture of the first and second premixes can then be cooled to about 45 C where other ingredients such as amphoteric surfactants, fragrance, preservative, opacifying materials, and so forth. The mixture can then be allowed to further cool to room temperature.

### 10. Examples

The following examples in Tables 1 and 2 are formulated as described below. Making instructions for Examples 1-10 can be found in the second paragraph of section 9 of this application. QS means quantity sufficient to reach 100%. All values are percent by weight.

TABLE 1

| | Example | | | | |
|---|---|---|---|---|---|
| | 1 | 2 | 3 | 4 | 5 |
| Sorbitol 70% Solution | 0.9715% | 0.9715% | 0.4857% | 0.9715% | 0.9715% |
| Glycerin | 0.4857% | 0.4857% | 0.9715% | 0.4857% | 0.4857% |
| Hydroxyethyl cellulose¹ | 0.4857% | 0.4857% | 0.4857% | 0.4857% | 0.2429% |
| PEG-90M² | 0.1652% | 0.1652% | 0.1214% | 0.1652% | 0.1943% |
| PEG-23M³ | 0.0486% | 0.0486% | 0.0729% | 0.0486% | 0.0486% |
| PTFE⁴ | 0.1457% | 0.1457% | 0.0972% | 0.1457% | |
| Palmitic acid | 7.5291% | 7.5291% | 7.5291% | 7.5291% | 7.5291% |
| Stearic Acid | 2.5259% | 2.5259% | 2.5259% | 2.5259% | 2.5259% |
| Glyceryl Oleate | 1.9430% | 1.9430% | 1.7532% | 1.9430% | 1.9430% |
| Triethanolamine (99%) | 5.8776% | 5.8776% | 5.8776% | 5.8776% | 5.8776% |
| Lubrajel Oil⁵ | 0.9715% | 0.9715% | 0.4857% | 0.9715% | 0.9715% |
| Fragrance | 0.7772% | 0.7772% | 0.7772% | 0.7772% | 0.6257% |
| Dye | 0.0029% | 0.0029% | 0.0029% | 0.0029% | 0.0035% |
| Menthol | 0.1457% | 0.1749% | 0.1749% | 0.1749% | |
| SoftCAT SL-5⁶ | 0.1943% | 0.0972% | | | |
| (PQ67) | | | | | |
| SoftCAT SL-30⁶ | | | 0.0729% | | |
| (PQ67) | | | | | |
| SoftCAT SL-60⁶ | | | | 0.0486% | 0.0486% |
| (PQ67) | | | | | |
| Isopentane (and) | 2.8500% | 2.8500% | 2.8500% | 2.8500% | 2.8500% |
| Isobutane | | | | | |
| Water | QS | QS | QS | QS | QS |

TABLE 2

| | Example | | | | |
|---|---|---|---|---|---|
| | 6 | 7 | 8 | 9 | 10 |
| Sorbitol 70% Solution | 0.9715% | 0.9715% | 0.9715% | 0.9715% | 0.9715% |
| Glycerin | 0.4857% | 0.9715% | 0.4857% | 0.4857% | 0.4857% |
| hydroxyethyl cellulose1 | 0.4857% | 0.4857% | 0.4857% | 0.9715% | 0.4857% |
| PEG-90M² | 0.1652% | 0.1652% | 0.1652% | 0.1214% | 0.1652% |
| PEG-23M³ | 0.0486% | 0.0729% | 0.0486% | 0.0486% | 0.0486% |
| PTFE⁴ | 0.1457% | 0.0972% | 0.1457% | 0.1457% | 0.1457% |
| Palmitic acid | 7.5291% | 7.1527% | 6.7762% | 6.7762% | 7.1522% |
| Stearic Acid | 2.5259% | 2.3996% | 2.2733% | 2.2733% | 2.3996% |
| Glyceryl Oleate | 1.9430% | 2.1540% | 1.9430% | 1.9430% | 1.9430% |
| Triethanolamine (99%) | 5.8776% | 5.5861% | 5.2898% | 5.2898% | 5.5861% |
| Lubrajel Oil⁵ | 0.9715% | 0.4857% | 0.9715% | 0.4857% | 0.9715% |
| Fragrance | 0.7772% | 0.7772% | 0.7772% | 0.8258% | 0.7772% |
| Dye | 0.0029% | 0.0029% | 0.0029% | 0.0029% | 0.0029% |
| Menthol | 0.1457% | 0.1312% | 0.1457% | | 0.1312% |
| SoftCAT SL-60⁶ | 0.0486% | 0.0243% | 0.0486% | 0.0972% | 0.0486% |
| (PQ67) | | | | | |
| Isopentane (and) | 2.8500% | 2.8500% | 2.8500% | 2.8500% | 2.8500% |
| Isobutane | | | | | |
| Water | QS | QS | QS | QS | QS |

1Available as Natrosol 250 HHR from Hercules Inc., Wilmington, DE
²Available as Polyox WSR-301 from Amerchol Corp., Piscataway, NJ
³Available as Polyox WSR N-12K from Amerchol Corp., Piscataway, NJ
⁴Available as Microslip 519 from Micro Powders Inc., Tarrytown, NY
⁵Available from Guardian Laboratories, Hauppauge, NY
⁶Available from The Dow Chemical Company, Midland, MI

Samples of Examples 11-20 are made as follows.
a. Weigh out a portion of water into a vessel sufficiently large enough to hold the entire batch. Mix with an overhead-mixer and impeller to create a vortex.
b. Add the cationic polysaccharide, hydroxyethylcellulose and disodium EDTA.
c. Heat the batch to about 75 C to hydrate the polymers, increasing rpms to maintain adequate mixing.
d. Prepare a separate premix by dispersing hydroxypropyl methylcellulose into water and neutralizing with triethanolamine or potassium hydroxide until the mixture becomes clear and thickens.
e. Slowly add the premix in d. to the batch in c. taking care to maintain a temperature about 75 C to minimize polymers from falling out of solution. Continue heating and mixing until well blended.
f. Add fatty acids and mix until well blended.
g. Prepare a separate premix by dispersing PEG 23M in glycerin and add to the batch in f.
h. Cool to below about 45 C and add amphoteric surfactants, fragrance, preservative, and, if desired, opacifying materials such as mixtures containing titanium dioxide.
i. Cool to below 35 C and QS with water.

TABLE 3
Examples with anionic soap and amphoteric surfactants
which are clear or translucent. Samples 11-13 are made
according to the below formulations.

| Ingredient | 11 | 12 | 13 |
|---|---|---|---|
| Water | QS | QS | QS |
| Triethanolamine | 4.25% | 4.25% | 4.25% |
| Lauric Acid | 1.50% | 1.50% | 4.50% |
| Myristic Acid | 4.50% | 4.50% | 1.50% |
| Cocamidopropyl Betaine (Mackam 35) | 7.14% | | |
| Lauramidopropyl Betaine (Mackam | | 10.00% | 10.00% |
| DAB-ULS) | | | |
| Glycerin | 4.00% | 4.00% | 2.00% |
| Hydroxyethylcellulose (Natrosol 250 | 1.00% | 1.00% | 1.30% |
| HHR CS) | | | |
| PQ-67 (SoftCAT SL-5) | 0.45% | | 0.05% |
| PQ67 (SoftCAT SL-60) | | 0.45% | 0.25% |
| Hydroxypropyl Methylcellulose | 0.25% | 0.25% | 0.15% |
| (Methocel 40-101) | | | |
| PEG 23M | 0.20% | 0.20% | 0.20% |
| Disodium EDTA | 0.05% | 0.05% | 0.05% |
| Preservative - Glydant Plus | 0.32% | 0.32% | 0.32% |
| Fragrance | 0.75% | 0.85% | 0.85% |

TABLE 4
Examples with anionic soap and amphoteric surfactants which are opaque.
Examples 14-20 are made according to the below formulations.

| Ingredient | 14 | 15 | 16 | 17 | 18 | 19 | 20 |
|---|---|---|---|---|---|---|---|
| Water | QS | QS | QS | QS | QS | QS | QS |
| Potassium Hydroxide | 1.60% | 1.60% | 1.60% | 1.60% | 1.95% | 1.60% | 1.95% |
| (45% aqueous) | | | | | | | |
| Lauric Acid | 3.00% | 1.50% | 1.50% | 1.50% | 1.50% | 3.00% | 1.50% |
| Myristic Acid | 1.50% | 3.00% | 3.00% | 3.00% | 1.50% | 1.50% | 3.00% |
| Palmitic Acid/Stearic | 1.50% | 1.50% | 1.50% | 1.50% | 4.50% | 1.50% | 3.00% |
| Acid (Emersol 132 NF) | | | | | | | |
| Lauramidopropyl | | | | | | 10.00% | |
| Betaine (Mackam | | | | | | | |
| DAB-ULS) | | | | | | | |
| Coco-Betaine (Mackam | 7.14% | 10.00% | 10.00% | 10.00% | 7.14% | | 10.00% |
| CB35-ULS-HP) | | | | | | | |
| Glycerin | 2.00% | 4.00% | 4.00% | 4.00% | 2.00% | 2.00% | 4.00% |
| Hydroxyethylcellulose | 0.20% | 0.40% | 0.40% | 0.40% | 0.20% | 0.30% | 0.40% |
| (Natrosol 250 HHR | | | | | | | |
| CS) | | | | | | | |
| Polyquaternium-67 | 0.45% | | | | 0.05% | | |
| (SoftCAT SL-5) | | | | | | | |
| Polyquaternium-67 | | 0.10% | 0.05% | 0.05% | | 0.10% | 0.05% |
| (SoftCAT SL-60) | | | | | | | |
| Hydroxypropyl | 0.15% | 0.25% | 0.25% | 0.25% | 0.35% | 0.25% | 0.25% |
| Methylcellulose | | | | | | | |
| (Methocel 40-101) | | | | | | | |
| PEG 23M | 0.20% | 0.20% | 0.60% | 0.60% | 0.40% | 0.60% | 0.60% |
| TiO2/Water/Glycerin/ | | | | 0.375% | 0.25% | 0.375% | 0.25% |
| Ammonium | | | | | | | |
| Polyacrylate | | | | | | | |
| (GLW75PFAP-MP) | | | | | | | |
| Disodium EDTA | 0.05% | 0.05% | 0.05% | 0.05% | 0.05% | 0.05% | 0.05% |
| Preservative - Glydant | 0.32% | 0.32% | 0.32% | 0.32% | 0.32% | 0.32% | 0.32% |
| Plus | | | | | | | |
| Fragrance | 0.75% | 0.75% | 0.85% | 0.85% | 0.85% | 0.85% | 0.85% |

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” All measurements are performed at 23° C., unless otherwise specified.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a,” “an,” and “the mean one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a. from about 0.005% to about 5% of a cationic polysaccharide, wherein said cationic polysaccharide is hydrophobically modified with a hydrophobic substituent and a cationic substituent;
b. from about 2% to about 25% of a water dispersible surface active agent; and
c. from about 60% to about 93% of a carrier comprising water.

2. The personal care composition of claim 1, further comprising from about 1% to about 6% of a volatile post-foaming agent.

3. The personal care composition of claim 1 wherein the personal care composition is a post foaming shave gel or an aerosol foam.

4. The personal care composition of claim 1, wherein the level of said hydrophobically modified cationic polysaccharide is from about 0.01% to about 2.0%.

5. The personal care composition of claim 1, wherein the hydrophobically modified cationic polysaccharide comprises a cellulose ether which has from 4,000 to 10,000 anhydroglucose repeat units and which is substituted with
(a) on the average from 0.0003 to 0.08 moles, per mole of anhydroglucose unit, of a substituent comprising an alkyl or arylalkyl group having from 8 to 24 carbon atoms and
(b) a substituent having the formula [R⁵R⁶R⁷R⁸N⁺](A^{z-})_{1z}
wherein R⁵, R⁶ and R⁷ each independently are —CH₃ or —C₂H₅, R⁸ is CH₂—CHOH—CH₂— or —CH₂CH₂—, A^{z-} is an anion, and z is 1, 2 or 3.

6. The personal care composition of claim 5, wherein said hydrophobically modified cationic polysaccharide comprises a cellulose ether selected from the group consisting of: hydroxyethyl cellulose, hydroxypropyl cellulose, methyl cellulose, hydroxypropyl methyl cellulose, hydroxyethyl methyl cellulose, hydroxyethyl carboxylmethyl cellulose, or mixtures thereof.

7. The personal care composition of claim 6, wherein said cellulose ether comprises a hydroxyethyl cellulose.

8. The personal care composition of claim 7, wherein said cellulose ether comprises on the average from 1.0 to 3.0 moles of hydroxyethyl groups per mole of anhydroglucose unit.

9. The personal care composition of claim 7, wherein said cellulose ether comprises on the average from 0.0005 to 0.07 moles of the substituent (a), per mole of anhydroglucose unit.

10. The personal care composition of claim 7, wherein said cellulose ether comprises on the average from 0.1 to 0.6 moles of the substituent (b), per mole of anhydroglucose unit.

11. The personal care composition of claim 7, wherein the substituent (a) has the formula: R¹R²R³R⁴N⁺(A^{z-})_{1z}
wherein R¹ and R² each independently are —CH₃ or —C₂H₅, R³ is —CH₂—CHOH—CH₂— or —CH₂CH₂—, R⁴ is an alkyl or arylalkyl group having from 8 to 24 carbon atoms, and A^{z-} is an anion, and z is 1, 2 or 3.

12. The personal care composition of claim 1, further comprising a lubricant, said lubricant comprises at least one of: a lubricious water soluble polymer, a water insoluble particle, a hydrogel-forming polymer, and a mixture thereof.

13. The personal care composition of claim 1, wherein the lubricant comprises at least one of: from about 0.01% to about 1%, of a lubricious water soluble polymer, from about 0.01% to about 5% of a water insoluble particle, from about 0.0005% to about 3%, of a hydrogel-forming polymer, and a mixture thereof, by weight.

14. The personal care composition of claim 12, wherein said lubricious water soluble polymers comprises at least one of a polyethylene oxide, a polyvinylpyrrolidone, a polyacrylamide, and a mixture thereof.

15. The personal care composition of claim 12, wherein said water insoluble particles comprises at least one of an inorganic particles, an organic polymer particles, and a mixture thereof.

16. The personal care composition of claim 12, wherein said hydrogel-forming polymers comprises at least one of: a polyacrylic acid or polymethacrylic acid partially esterified with a polyhydric alcohol; a hydrophilic polyurethanes; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

17. The personal care composition of claim 16, wherein said hydrogel-forming polymer comprises polyacrylic acid partially esterified with glycerin.

18. The personal care composition of claim 1, wherein said water dispers able surface active agent comprises a lathering surfactant other than a non-ionic surfactant.

19. The personal care composition of claim 1, wherein said water dispersable surface active agent is free or essentially free of a soap.

20. A method of making a personal care composition according to claim 1, comprising:
a. forming a first premix comprising water and a water soluble polymers, said water soluble polymer comprising a cationic polysaccharide, and optionally a polyethylene oxide, hydroxyethylcellulose, or a mixture thereof;
b. forming at least a second premix comprising a hydroxypropylmethylcellulose, a base and water; and
c. combining said first premix and said second premix.
